(19) United States
(12) Patent Application Publication
(10) Pub. No.: US 2013/0065195 A1
(43) Pub. Date: Mar. 14, 2013

(54) METHOD FOR MANUFACTURING A TEMPLATE FOR PROVIDING DENTAL IMPLANTS IN A JAW AND SCAN PROSTHESIS FOR APPLYING THIS METHOD

(71) Applicant: René De Clerck, Tervuren (BE)

(72) Inventor: René De Clerck, Tervuren (BE)

(73) Assignee: DENTAL VISION BVBA, Tervuren (BE)

(21) Appl. No.: 13/699,416

(22) PCT Filed: May 23, 2011
(86) PCT No.: PCT/BE2011/000032

§ 371 Date: Nov. 21, 2012

(30) Foreign Application Priority Data
May 21, 2010 (BE) 2010/0307

(51) Int. Cl. A61C 8/00; A61B 6/14

(52) U.S. Cl. 433/29; 433/173

(57) ABSTRACT

The invention concerns a method for manufacturing a template with at least one opening to drill through so as to create an appropriate bore hole in the bone of an at least partially toothless lower or upper jaw so as to fix an implant in said bone.

The invention concerns a method for manufacturing a template with at least one opening to drill through so as to create an appropriate bore hole in the bone of an at least partially toothless lower or upper jaw so as to fix an implant in said bone. After an implant has been fixed in the bore hole in the jaw, a dental prosthesis must be mounted on the latter.

Implants are mainly provided in a jaw in order to anchor dental prostheses. Such prostheses may consist for example of a solitary false tooth which is fixed on a single implant or of a superstructure which extends over a major part of the jaw and on which several false teeth are provided. Also, such a superstructure is mounted on several implants, i.e. two, three, four or more implants.

The aim is now to provide a patient with a dental prosthesis in as little as treatment steps as possible. The implants are hereby immediately loaded, as they say, whereby a permanent dental prosthesis is mounted on these implants practically while the implants are being provided in the jaw. Such a method requires a thorough planning and a very high accuracy when providing the implants. Indeed, the planned position of the implants to be provided must correspond practically perfectly with the actual definite position of the implants after they have been provided in the jaw. Indeed, the dental prosthesis is made on the basis of the planned position of the implants before the latter are actually provided in the jaw.

In order to make this possible, use is made of a template according to the present state of the art to precisely drill bore holes in which the implants are to be fixed. However, many of the existing techniques are not patient-friendly or they are rather complex and do not provide sufficient guarantees as to obtaining the required accuracy.

Thus, the invention aims to provide a method for manufacturing a template which is practically free of any discomforts for the patient, whereby a template is obtained which makes it possible to provide the planned bore holes for implants in a jaw in a very accurate manner.

To this aim, according to the method of the invention, a scan prosthesis with provisional prosthetic teeth is made provided with at least one three-dimensional means of reference.

Next, a first three-dimensional image, in particular a three-dimensional X-ray is made of the jaw together with the scan prosthesis and said three-dimensional means of reference in order to determine critical anatomical structures of the jaw.

In addition to this, a second three-dimensional image of visible surfaces of present teeth and/or of the jaw together with the scan prosthesis and said three-dimensional means of reference is generated.

The data of the first and second three-dimensional images are correlated by means of said means of reference.

Further, in this method, the position for the corresponding bore hole for providing an implant is selected on the basis of the detected position of said critical anatomical structures and the position of said provisional prosthetic teeth. This position of the bore hole is defined in relation to the three-dimensional means of reference in said first three-dimensional image.

According to the invention, a template is made then which can be applied precisely fitting on teeth which are present in the jaw concerned in the oral cavity. In this template, said opening is provided in such a position in relation to said three-dimensional means of reference that, when this template is applied in a fitting manner on teeth present in the oral cavity, the position of this opening makes it possible, when drilling through this opening, to form said bore hole in correspondence with the selected position.

Advantageously, a physical model of the jaw is made and said second three-dimensional image is generated on the basis of this physical model when said scan prosthesis, provided with said means of reference, is applied in a fitting manner on this physical model.

According to a preferred embodiment of the method according to the invention, several reference elements are used for said three-dimensional means of reference which are mutually connected in a predetermined position.

On the basis of the data of said second three-dimensional image, the negative form of at least the occlusion surface of the teeth which are present in the oral cavity is preferably provided in the template, such that the latter can be provided in a precisely fitting manner on these teeth in the oral cavity.

The invention also concerns a scan prosthesis to be used with the method according to the invention, whereby this scan prosthesis contains false teeth made of a material which is clearly visible in an X-ray, whereby this scan prosthesis contains at least three marking elements made of a highly contrasting material in X-rays.

In this scan prosthesis, said marking elements preferably extend above the occlusion surface of said false teeth and they are mutually situated in a predetermined calibrated position.

The scan prosthesis is made in a way known to the expert. In general, a physical model is usually made of the jaw, in particular a plaster model, and provisional prosthetic teeth are put in this model. The latter can be done for example by putting the physical model in an articulator and by fixing the teeth on a base by means of what is called the lost-wax technique, which base is applied on the model in a fitting manner. The false teeth themselves can be made of a material which is clearly visible in an X-ray thereof, such as for example barium sulphate.

This base with the provisional prosthetic teeth then forms the scan prosthesis which can be put in the oral cavity in a fitting manner in the position where implants are to be provided on which a dental prosthesis will be mounted. Thus, the shape of these prosthetic teeth and their position normally corresponds with that of the permanent dental prosthesis.

Possibly, in an interesting manner, this scan prosthesis is provided with additional supporting means so as to make it rest in a precisely fitting manner on adjacent teeth present in the oral cavity. These supporting means may have a surface which has the negative form of for example the occlusion surface of said adjacent teeth.

According to the method of the invention, at least one three-dimensional means of reference is fixed to this scan prosthesis.

This means of reference is preferably selected such that its position can be clearly determined in a roentgenogram made thereof. Such a means of reference consists for example of a support which is transparent to X-rays and in which have been provided three or more marking elements. These marking elements are clearly visible in a roentgenogram and preferably have a convex volume which is made for example of barium sulphate, gutta percha, tantalum, platinum or tungsten.

In order to improve the accuracy of a position determination of the means of reference in an image, in particular in an X-ray, the marking elements are preferably connected in relation to one another in a predetermined position, such that their mutual position is precisely known.

Thus, the marking elements may for example be each embedded in a reference element which thus forms a support for one or several marking elements. These reference elements are formed for example of a cylindrical rod which thus comprises at least one marking element, whereby the reference elements are advantageously mounted on a jig plate, such that the relative position of the reference elements, and consequently of the accompanying marking elements, is precisely determined. Next, the reference elements are firmly connected to the scan prosthesis. Once the reference elements are fixed to the scan prosthesis, the jig plate may be possibly removed.

Consequently, when an image is thus made of the scan prosthesis with the reference elements, the position and orientation of the reference elements can be precisely determined on the basis of the observed position of the marking elements, also taking into account the position of the provisional prosthetic teeth.

In order to obtain a clear image in a roentgenogram, it is preferably made sure that said reference elements are connected to the scan element in such a manner that they extend at least above the occlusion surface of said provisional prosthetic teeth. In particular, said marking elements advantageously extend above the occlusion surface of said provisional prosthetic teeth.

According to the invention, a first three-dimensional image, in particular a three-dimensional X-ray is made of the jaw together with the scan prosthesis and said three-dimensional means of reference in order to determine critical anatomical structures of the jaw, such as the position of nerve bundles and the available bone structure. In this image is preferably also observed the position of said provisional prosthetic teeth. Such a three-dimensional image is generated for example by means of what is called a CT-scan. Thus, the position of critical anatomical structures is determined in relation to said means of reference or, in other words in relation to said marking elements. As the mutual position of the marking elements is calibrated and thus predetermined, the accuracy of this position determination of the critical anatomical structures is considerably improved.

In addition to this, also a second three-dimensional image of visible surfaces of teeth that are present in the jaw is generated, and possibly of visible elements of the jaw itself, together with the scan prosthesis and said three-dimensional means of reference.

This second three-dimensional image must not necessarily be made in the oral cavity but is preferably generated on the basis of said physical model when the scan prosthesis has been applied on the latter in a fitting manner together with the means of reference. The second three-dimensional image can be obtained for example by means of a laser scan. Data regarding the shape of the teeth present, in particular their occlusion surfaces, can then be derived from this second image, as well as the position and orientation of the means of reference, in particular of said reference elements and/or the position of the marking elements.

Next, the data of the first and second three-dimensional images are correlated on the basis of said means of reference. In particular, these images or their data are superimposed, whereby the means of reference from both images are made to fit precisely on one another. This implies in particular that the images of the three-dimensional means of reference are made to coincide in both images. Indeed, the determination of the reference elements in both images makes it possible to make the data of the first and second three-dimensional images correlate very precisely.

On the basis of the observation of the critical anatomical structures and of said provisional prosthetic teeth in the first three-dimensional image is selected a position for the corresponding bore hole for an implant to be provided.

The external shape and position of the teeth which are present in the oral cavity can then be correlated to the selected position for the bore hole, such that a template can be made for the latter which can be applied in a precisely fitting manner on the teeth present in the oral cavity.

Thus is made a template which can be applied on teeth present in the jaw in the oral cavity in a precisely fitting manner. Said opening in the template is hereby provided in such a position that, when the template has been applied on teeth present in the oral cavity, the position of this opening makes it possible, when drilling through this opening, to create said bore hole in correspondence with the selected position.

In particular, on the basis of the data of said second three-dimensional image, the negative form of at least the occlusion surface of teeth present in the oral cavity is provided in the template, such that the latter can be applied in a fitting manner on these teeth in the oral cavity.

Thus, for example, on the basis of said second image, at least the highest point of the occlusion surface is determined for a number of teeth. Furthermore, the position of an extreme point of at least a lateral side of these teeth is preferably determined. This extreme point usually coincides with the place where the tooth has a maximum thickness. Thus, for a solitary tooth, the position of the top of at least one cuspid can be determined, for example, and of an extreme point on the vestibular, mesial and/or distal tooth surface and/or on the lingual or palatal tooth surface.

Normally, for adjacent teeth, at least the mesial or the distal tooth surface is not accessible, and normally, apart from determining the position of the tops of a number of cuspids, determining the position of a number of points on the palatal or lingual and the vestibular sides of the teeth will suffice.

Supports or bearing surfaces are provided on the template corresponding with the points whose position was determined on the teeth. In this manner is provided a recess in the template in which fits at least the occlusion surface of the teeth, and the template can be applied in a precisely fitting, stable manner on the teeth present in the jaw.

The number of points whose position is determined on the teeth depends on the number of teeth that is still present in the jaw and on the position of these teeth. In particular, the number of points whose position on the teeth is determined and the location of these points are selected such that the template can rest in a stable, precisely fitting manner on these points, whereby the template is prevented from shifting while a bore hole is being drilled in the jaw after it has been applied in a fitting manner on the teeth.

Naturally, in a number of cases it may be sufficient to determine only the position of a number of points in the occlusion surface, whereby a template is manufactured which fits precisely on these points of the occlusion surface. In other cases, it may possibly be sufficient to manufacture a template which only rests on points of the lateral tooth surfaces of the teeth present.

Although it was described above how the position of points on the teeth in the jaw is determined for manufacturing a template with corresponding supports or bearing surfaces, it is clear that it is also possible to select surfaces on the teeth and to precisely define them by means of the above-mentioned second three-dimensional image. Corresponding supports or bearing surfaces are then provided on the template which must fit tightly to and rest on said defined surfaces of the teeth.

The template is preferably made by means of a CAD/CAM production process, in particular a “Rapid prototyping” technique such as milling, selective laser sintering or melting, stereolithografy, 3D printing, etc.

Sometimes, there are insufficient teeth in the jaw concerned to apply a template in a stable manner. In this case, a supporting element such as a small screw may possibly be provided in the jaw's bone. Means are then provided on the template to be fixed on this supporting element or to rest on it.

In order to drill said bore hole, the template is applied in a fitting manner on the teeth present in the jaw concerned in the oral cavity, and drilling is subsequently done through the opening concerned. Guide bushes may be provided in the opening of the template to guide the drill. An implant can then be screwed through the opening of the template in the bore hole.

Of course it is possible to manufacture a template according to the method of the invention which is provided with one, two or several holes for drilling a bore hole in a jaw to provide a corresponding number of implants.

The method according to the invention thus allows to provide one or several implants with very high accuracy in a predetermined position in the jaw. Thus it is possible to mount a superstructure on the implants, immediately after they have been provided in the jaw, which has been made on the basis of the predetermined position of the implants.

1. Method for manufacturing a template with at least one opening to drill through so as to create an appropriate bore hole in the bone of an at least partially toothless lower or upper jaw for fixing an implant in said bone in order to mount a dental prosthesis on this implant,
whereby
a scan prosthesis with provisional prosthetic teeth is manufactured;
this scan prosthesis is provided with at least a three-dimensional means of reference;
a first three-dimensional image, in particular a three-dimensional X-ray, is made of the jaw together with the scan prosthesis and said three-dimensional means of reference in order to determine critical anatomical structures of the jaw;
a second three-dimensional image is generated of visible surfaces of teeth present together with the scan prosthesis provided with said three-dimensional means of reference;
said first and second three-dimensional images are correlated by means of said means of reference;
on the basis of the determined position of said critical anatomical structures and the position of said provisional prosthetic teeth, the position for the corresponding bore hole is selected;
a template is manufactured, whereby bearing surfaces and/or supports are provided on this template on the basis of said second image of visible surfaces of teeth present, such that the template can be applied in a fitting manner via said bearing surfaces and/or supports on these teeth present;
said opening in the template is provided in such a position that, when this template has been applied in a fitting manner on teeth present in the oral cavity, the position of this opening makes it possible, when drilling through this opening, to create said bore hole in correspondence with the selected position.

2. Method according to claim 1, whereby a physical model of the jaw is made and said second three-dimensional image is generated from this physical model when said scan prosthesis provided with said means of reference has been applied in a fitting manner on this physical model.

3. Method according to claim 1 or 2, whereby several reference elements which are mutually connected in a predetermined position are used for said three-dimensional means of reference.

4. Method according to claim 1 or 2, whereby said three-dimensional means of reference comprises several reference elements which are each fixed to said scan prosthesis in a separate position.

5. Method according to claim 3 or 4, whereby said reference elements are connected to the scan prosthesis in such a manner that they extend at least above the occlusion surface of said provisional prosthetic teeth.

6. Method according to any one of claims 3 to 5, whereby each of said reference elements is provided with a marking element made of a material which is highly contrasting in said first three-dimensional image.

7. Method according to claim 6, whereby said marking element extends above the occlusion surface of said provisional prosthetic teeth.

8. Method according to any one of claims 1 to 7, whereby on the basis of said second image, for at least a number of teeth, the position of an extreme point of at least one lateral side of these teeth is determined, whereby a corresponding support or bearing surface is provided in a recess of the template for these extreme points, such that the template can fit tightly via said support or bearing surfaces to these extreme points of the corresponding teeth, whereby said recess extends over these teeth.

9. Method according to any one of claims 1 to 8, whereby on the basis of said second image for at least a number of teeth, the top of a cuspid of these teeth is determined, whereby a support or bearing surface is provided on the template which corresponds to said top of the teeth concerned, such that the template, via said support or bearing surface, can fit tightly onto the top of the cuspid of the corresponding teeth.

10. Method according to any one of claims 1 to 9, whereby said template is shaped such that it can be applied precisely fitting on any teeth present.

11. Method according to any one of claims 1 to 10, whereby on the basis of the data of said second three-dimensional image, the negative form of at least the occlusion surface of the teeth present in the oral cavity is provided in the template, such that the latter can be applied in a fitting manner on these teeth in the oral cavity.

12. Method according to any one of claims 1 to 10, whereby said template is manufactured by means of a CAD/CAM production process, in particular a “Rapid prototyping” technique.

13. Method according to any one of claims 1 to 11, whereby a three-dimensional object is selected for said three-dimensional means of reference.

14. Method according to any one of claims 1 to 13, whereby means are provided on said template in order to be fixed to a reference element which is provided in said jaw.

15. Method according to any one of claims 1 to 14, whereby said provisional prosthetic teeth are made of a material which is opaque in X-rays such as barium sulphate.

16. Scan prosthesis to be used with the method according to any one of claims 1 to 15, whereby it comprises false teeth made of a material which is clearly visible in an X-ray, whereby they are provided with at least three marking elements made of a highly contrasting material in X-rays.

17. Scan prosthesis according to claim 16, whereby said marking elements extend above the occlusion surface of said false teeth and whereby they are mutually positioned in a predetermined calibrated position.
